(19) United States
(12) Patent Application Publication
(10) Pub. No.: US 2003/0147937 A1
(43) Pub. Date: Aug. 7, 2003

(54) Use of compatible solutes as substances having free radical scavenging properties

(76) Inventor: Thomas Schwarz, Leichlingen (DE)

Correspondence Address: FULBRIGHT AND JAWORSKI L L P, PATENT DOCKETING 29TH FLOOR, 865 SOUTH FIGUEROA STREET, LOS ANGELES, CA, 900172576

(21) Appl. No.: 10/257,225

(22) PCT Filed: Apr. 6, 2001
(86) PCT No.: PCT/EP01/03935

(30) Foreign Application Priority Data
Apr. 12, 2000 (DE) 10018225.9

(51) Int. Cl. A61K 48/00; A61K 38/00; A61K 31/7024; A61K 31/66

(52) U.S. Cl. 424/439; 514/2; 514/44; 514/23; 514/102

(57) ABSTRACT

The invention relates to the use of compatible solutes selected from the group comprised of di-myo-inositol phosphate (DIP), cyclic 2,3 diphosphoglycerate (cDPG), 1,1-di-glyccrol phosphate (DGP), β-mannosylglycerate (firoin) β-mannosylglyceramide (firoin-A) and/or di-mannosyl-di-inositol phosphate (DMIP) and/or of derivatives of these compounds or combinations thereof for protecting organisms, organs, tissues, cells or the organic building blocks thereof from chemical radicals and oxidatively active compounds.

The invention relates to free radical scavenging or antioxidative compounds and compositions for treating disease and protecting the skin, biologic molecules and structures as well as foodstuffs, which compounds and compositions contain, as active components, various low-molecular substances (compatible solutes) from extremophilc microorganisms, notably di-myo-inositol phosphate (DIP), cyclic 2,3 diphosphoglycerate (cDPG). 1,1-di-glycerol phosphate (DGP), β-mannosylglycerate (firoin), β-mannosylglyceramide (firoin-A) or/and di-mannosyl-di-inositol phosphate (DMIP) and/or a derivate, e.g. an acid, salt or ester of these compounds as substances with free radical scavenging properties.

According to the invention, di-myo-inositol phosphate (DIP), cyclic 2,3 diphosphoglycerate (cDPG), 1,1-di-glycerol phosphate (DGP), β-mannosylglycerate (firoin), β-mannosylglyceramide (firoin-A) or/and di-mannosyl-di-inositol phosphate (DMIP) are useful, for example for protecting the skin against environmental effects (e.g. in cosmetology and dermatology), for treating disease, for protecting a biological material and for slowing down aging processes caused by free radical and oxidant activity.

### STATE OF THE ART

Formation of Free Radicals

Free radicals are atoms, ions or molecules that contain one or several unpaired electrons in their outer electron shell. Owing to this physiochemical characteristic, free radicals are unstable, highly reactive, energy-rich intermediates.

Reactions with oxygen are the basis for the formation of many free radicals. Active oxygen causes in vivo the formation of superoxide radicals, hydrogen peroxide, hydroxyl radicals and excited singlet oxygen. These types of radicals are called oxygen radicals or reactive oxygen species (ROS). Oxygen radicals are degraded in vivo by enzymes or scavenged and converted by natural free radical scavenging compounds, such as ascorbic acid. Superoxide is converted by dismutase. Hydrogen peroxide is removed y catalase and peroxidase. Singlet oxygen is degraded by beta-carotene and tocopherol.

Hydroxyl radicals are formed in vivo by the reaction of hydrogen peroxide with superoxide radicals. Hydroxyl radicals are highly reactive and react very quickly and in an uncontrolled manner with cell components. The duration of a single hydroxyl radical's existence is so short that organisms have not developed a mechanism to remove hydroxyl radicals.

In addition to the highly reactive radicals, such as the hydroxyl radical, there are moderately reactive radicals (superoxide radical) and persistent radicals (ascorbyl).

Typical representatives of oxygen radicals are:

Superoxide anion, O⁻₂ hydroxyl radical, lipid peroxide radical, radical oxygen, hydrogen peroxide as well as hypochlorous acid.

Toxic oxygen radicals are produced in a side reaction during respiration.

Furthermore, free radicals are produced in vivo by ultraviolet radiation (especially UV B), cigarette smoke, ozone or ionizing radiation (e.g. during radiation therapy for cancer treatment, x-ray examination). Consumption of alcoholic beverages and stress also lead to increased formation of radicals. In addition, radicals are formed due to reactions involving environmental poisons (e.g. herbicides, pesticides, solvents), heavy metals or petrochemicals. Even pharmaceutical substances and certain ingredients of foodstuffs have been described as free radical forming agents.

Natural Effects of Radical Reactions

According to many clinical reports, active oxygen and free radicals damage, for instance, the membrane tissue of a living body, thus causing various diseases.

Cells are oxidized (“oxidative stress”) due to free radical activity. This leads to new radicals being formed in a chain reaction; these new radicals, in turn, have a cell-damaging effect. Radicals are also formed when tissue is temporarily cut off from the oxygen supply via the blood as a result of a cardiac infarction, an apoplectic stroke or an organ, transplant, for example. Formation of free radicals also leads to irreversible damage to biological material during storage, even at low temperatures.

Free radicals damage and destroy cells by changing and destroying their natural components, e.g. cell proteins, lipoproteins, and lipids. Moreover, radical reactions damage also nucleic acids, which causes mutations in the DNA. Free radicals react irreversibly with free amino acids, carbon hydrates and tissue-forming macromolecules (e.g. collagen). Cell compartments, such as mitochondria, are attacked by free radicals. Thus free radicals react in an uncontrolled manner with molecules and macro-molecular structures of all biochemical classes.

Radical reactions lead to aging processes in biological structures. Radical oxidation of natural structures is analogous to the corrosion of metals. Thus dry skin or age spots are produced by free radical activity. Ageing of the skin is accelerated by radicalizing UV B and UV A radiation.

Stress (caused for example be jetlag) leads to increased formation of free radicals.

Generally, free radicals are toxic and cause numerous diseases and deficiency symptoms. To date, up to about 60 diseases have been described which are entirely or partly due to free radical activity. These diseases include AIDS, allergies, angina, arthritis, asthma, arteriosclerosis, internal bleeding, bleeding gums, hematomas, cancer, cataracts, circulation problems, cirrhosis, diabetes type 2, dehydration of the skin, edemas, fatigue, hay fever, heart attack, hemorrhoids, hypertension, inflamed tissues, liver and kidney damage, impotence, loss of memory, menstrual disorders, migraine, multiple sclerosis, night blindness, Parkinson's disease, phlebitis, prostate problems, psoriasis, respiratory problems, retinopathy, senility, rheumatism, skin cancer, skin disorders, apoplectic stroke, swollen limbs, varicose veins, etc. It has been proved empirically that there is a connection between oxidative damage from free radicals and the development of neurodegenerative diseases, such as Huntington's disease (or HD), Parkinson's disease (or PD), Lou Gebrig's disease (or ALS) and Alzheimer's disease (or AD).

However, not all radical reactions are toxic. Ubiquitous nitrogen oxide radicals (NO) are in various ways involved in controlling neurological, vascular and immunological functions. Besides these positive effects, NO radicals have also noxious effects. Thus NO reacts with superoxide radicals to form peroxy nitrite, which is even more reactive.

Free Radical Scavenging Substances/Antioxidants

According to the state of the art the formation of free radicals is suppressed or prevented by the use of so-called free radical scavenging substances. Free radical scavenging substances react with free radicals, forming products that are no longer radical or at least less reactive than the original radicals. Free radical scavengers thus act as “lightning conductors” on free radicals. The free radical scavengers that are involved in oxidative reactions are also known as antioxidants or antioxidative substances.

Natural free radical scavengers/antioxidants are glutathione, vitamin A or its base substance beta-carotene, vitamin C or analogous substances (ascorbic acid) or precursors (sorbite), vitamin E, N-acetyl-L-cysteines. The free radical scavenging property of the hormone melatonin has been described in the literature.

Pycuogenols and proanthenols from vegetable extracts (tea, pine bark) are also antioxidants. Further radical scavenging extracts from plants contain flavenoids as free radical scavengers. Proanthenols, for example, are effective against aging processes.

Sugar and polyols are known to have free radical scavenging potential.

Carbon hydrogen compounds such as pentadecane derivates, fatty acid esters of baccatin, alpha-chlorinated carbonates, 1,5-anhydrofructose. Certain essential fatty acids, too, reduce radical related noxious effects.

Another category of antioxidative substances is the group of pyrrolpyrimidines. A major representative of this group is 5,5-dimethyl-1-pyrrolin-N-oxide (DMPO).

Nicorandil or sesamin have been described in the literature as active components of free radical scavenging compositions.

The compatible solutes glutamate, prolin, trehalose, mannitol, sorbitol, inositol and betain derivates and esters are described as more or less effective free radical scavengers.

Isoquercitrin, toxerutin, doxorubicin, dihydrorobinetin, hydroquinone and phenylenediamin derivates are also reported to be free radical scavengers.

Minerals like selenium and zinc are also used as antioxidants. Selenium acts also as a co-factor to cellular radical-neutralizing enzymes.

Enzymes that catalyze radical reactions with free radical scavenging substances are also used to prevent radical chain reactions. According to the state of the art, the following substances used: dioxygenase, monooxygenase, oxidase, hydroxylase, superoxide dismutase, glutathione peroxidase and reductase, ecatalase, catalse or thiol-specific antioxidative enzymes.

Certain proteins act also as endogenous natural free radical scavengers. These proteins include transferrin, lactoferrin, ceruloplasmin, albumin, haptoglobin-hemoplexin, and urate.

When applying free radical scavengers, it is important to make sure that the substances are used do not themselves form toxic and reactive radicals under the influence of the radicals. Thus ideal free radical scavengers are substances that have non-toxic, non-mutagenic, non-carcinogenic, non-antigenic and non-teratogenic effects.

Quantification of Free Radical Scavenging Potential

Numerous enzymatic and chemical test methods for the quantification of radical reactions have been described in the literature. For the purpose of standardizing and comparing the free radical scavenging potential of various substances, the radical protection factor (RPF) has been introduced as a means to determine the radical scavenging activity of antioxidant products. The principle of the RPF determination method is based on the electron spin resonance (ESR), which is measured with an ESR spectrometer. The test substance used is a highly reactive radical that reacts with all know antioxidants.

Application of Radical Scavenging Substances/Antioxidants

The application of free radical scavenging or antioxidant substances may reduce or even prevent the development of free radical related cell alterations and illnesses. Antioxidants or free radical scavengers boost the human body's defense system and have cancer and inflammation inhibiting effects.

At any rate, non-toxic radical scavengers reduce the risk of acquiring free radical induced diseases and increase the regenerative capacity of damaged cells and tissues. Natural, free radical induced processes, such as aging, can be decelerated.

Thus the potential for using antioxidants in medicine, cosmetology/dermatology and nutrition is manifold. According to the state of the art, vitamin A is used for skin protection. Vitamin C provides protection against the noxious effects of tobacco smoke and is effective against chronic bronchitis as well as other lung diseases. Vitamin C supports healing processes and stimulates cell regeneration. The application of vitamin E can reduce the heart attack risk. N-acetyl-L-cysteine minimizes the risk of cancer and abnormal coagulation inside of blood vessels. Selenium destroys free radicals and thus provides protection against cancer. Glutathione is involved in the reduction of toxic products of metabolism and eliminates free radicals. Thus glutathione minimizes the risk of heart disease, cancer, immune deficiency and nervous disease. Furthermore, glutathione has a controlling effect on other important antioxidants.

Free radical, scavengers and antioxidants are taken orally as tablets, capsules, granules or powder; they are used as carriers or excipients.

Accordingly, one objective of the invention is to provide a category of substances that has a direct effect on hydroxyl radicals and other radicals.

This objective is achieved by using compatible solutes selected from the group comprised of di-myo-inositol phosphate (DIP), cyclic 2,3 diphosphoglycerate (cDPG), 1,1-di-glycerol phosphate (DGP), β-mannosylglycerate (firoin), β-mannosylglyceramide (firoin-A) or/and di-mannosyl-di-inositol phosphate (DMIP) and/or derivates of these compounds or combinations thereof for protecting organisms, organs, tissues, cells or the buildings blocks thereof from chemical radicals and oxidatively active compounds.

Surprisingly enough, it was found that the compatible solutes di-myo-inositol phosphate (DIP), cyclic 2,3 diphosphoglycerate (cDPG), 1,1-di-glycerol phosphate (DGP), β-mannosylglycerate (firoin), β-mannosylglyceramide (firoin-A) or/and di-mannosyl-di-inositol phosphate (DMIP) have extremely good free radical scavenging potential and are thus good radical scavengers, prophylactics and medicines against disease, compositions for skin protection as well as protectors for biological material and food supplements.

The invention thus relates to the use of at least one substance selected from the group of the following compounds: di-myo-inositol phosphate (DTP), cyclic 2,3 diphosphoglycerate (cDPG), 1,1-di-glycerol phosphate (DGP), β-mannosylglyceramide (firoin), β-mannosylglyceramide (firoin-A) or/and di-mannosyl-di-inositol phosphate (DMIP) or a derivate of these compounds, e.g. an acid, salt or ester of these compounds for the production of a pharmaceutical or cosmetic preparation as well as preparations for the protection of biological molecules and structures as well as food products.

According to the invention, there is no specific limitation to the form of application of the composition. Examples of the form of application are: preparations for oral application or injection, preparations in the form of suppositories, preparations for external application (e.g. condition blistering plasters, ointments, lotions) or eye solutions.

Diseases and impairments of organs which are caused by free radicals or oxidants or in which free radicals or oxidants are implicated can be treated or prevented by using the substances specified in this invention.

The dose of the active component contained in each preparation as covered by the present invention is selected in accordance with the age, sex, and health condition of the specific patient or user.

The application of compatible solutes according to this invention protects in particular organic building blocks, such as proteins, lipids, fats or nucleic acids. The organisms protected are notably human beings, animals, and microorganisms. The organs that—according to the invention—can be protected by the compatible solutes from chemical radicals and oxidant substances are in particular tissue, skin, kidneys, heart, and limbs.

The compatible solutes are preferably applied in concentrations of 0.001 M to 2 M by introducing them, in an appropriate form, into the organisms, organs, tissues, cells—or the organic building blocks thereof—to be protected.

According to the invention, the compatible solutes can be used to produce a medicine to treat diseases that are caused by free radical and oxidant activity. Such diseases are in particular: heart diseases such as cardiac infarction, stroke, rejection after an organ transplant, AIDS, allergies, angina, arthritis, asthma, arteriosclerosis, internal bleeding, bleeding gums, hematomas, cancer, cataracts, circulation problems, cirrhosis, diabetes type II, edemas, fatigue, hay fever, heart attack, hemorrhoids, hypertension, inflamed tissues, liver and kidney damage, impotence, memory loss, menstrual disorders, migraine, multiple sclerosis, night blindness, Parkinson's disease, phlebitis, prostate problems, psoriasis, respiratory problems, retinopathy, senility, rheumatism, skin cancer, stroke, swollen limbs, varicose veins, as well as neurodegenerative diseases, such as Huntington's disease (or HD), Parkinson's disease (or PD), Lou Gelrig's disease (or ALS) and Alzheimer's disease (or AD).

Furthermore, the compatible solutes according to the invention can be used to produce a cosmetic and/or dermatological preparation for the prevention or treatment of skin damage or skin disorders caused by free radical and oxidant activity.

The terms skin damage and skin disorders cover in particular dehydration of the skin, skin problems, dermatoses, and age spots.

The compatible solutes can also be used for the production of a food product for the prevention and treatment of damage or alterations to organisms caused by free radical and oxidant activity.

The effectiveness of the preparation consisting of solutions of the compatible solute di-myo-inositol phosphate (DIP), cyclic 2,3 diphosphoglycerate (cDPG), 1,1-di-glycerol phosphate (DGP), β-mannosylglycerate (firoin), β-mannosylglyceramide (firoin-A) or/and di-mannosyl-di-inositol phosphate (DMIP) has been demonstrated by the following example:

100 mM of sodium phosphate buffer, pH 6.8, was used as buffer. For the quantitative determination of the di-myo-inositol phosphate (DIP), cyclic 2,3 diphosphoglycerate (cDPG), 1,1-di-glycerol phosphate (DGP), β-mannosylglycerate (firoin), β-mannosylglyceramide (firoin-A) or/and di-mannosyl-di-inositol phosphate (DMIP), a solution of each of these compounds was mixed with DPPH (2,2-bis-(4-(1,1,3,3-tetramethylbutyl)-phenyl)1-picrylhydracyl; final concentration: 0.125 mM).

Subsequently a determination was made to ascertain the RPF of di-myo-inositol phosphate (DIP), cyclic 2,3 diphosphoglycerate (cDPG), 1,1-di-glycerol phosphate (DGP), β-mannosylglycerate (firoin), β-mannosylglyceramide (firoin-A) or/and di-mannosyl-di-inositol phosphate (DMIP).

The RPF, determined by the method of Herrling et. al, Cosmetic World Journal (1998), shows the number of test radicals that are reduced by 1 mg of the substance used. The test radical used was DPPH.

Results

For the exact determination of the RPF, three readings were taken after defined reaction times (30 min., 3 h, and 24 h), and the time constant of the establishment of equilibrium was determined. In all cases, the equilibrium was almost completely established after 24 h. Thus the values measured at that time were used to determine the RPF. The RPF (protection factor against DPPH) of ascorbic acid-2-phosphate (an excellent radical scavenger) and of ectoin (a very poor radical scavenger) was determined as reference values (against DPPH). The results are shown in the following table.

Table: RPF Values

Potential for radical scavenging by di-myo-inositol phosphate (DIP), cyclic 2,3 diphosphoglycerate (cDPG), 1,1-di-glycerol phosphate (DGP), β-mannosylglycerate (firoin), β-mannosylglyceramide (firoin-A) or/and di-mannosyl-di-inositol phosphate (DMIP).

| | Concentration | Type of | Radical | Time | RPF |
|---|---|---|---|---|---|
| Sample | mg/ml | Radical | Conc. M | constant H | N/10¹⁴ |
| DIP | 5.1 | DPPH | 50.0 E-6 | 3.7 | 28 +/− 4 |
| Firoin | 9.0 | DFPH | 50.0 E-6 | 2.1 | 16 +/− 3 |
| Firoin-A | 12.0 | DPPH | 50.0 E-6 | 1.9 | 17 +/− 3 |
| DGP | 15 | DPPH | 50.0 E-6 | 2.4 | 24 +/− 3 |
| DM1P | 6 | DPPH | 50.0 E-6 | 3.6 | 27 +/− 4 |
| cDPG | 17.9 | DPPH | 50.0 E-6 | 2.9 | 9 +/− 0.3 |
| Ectoin | 60.3 | DPPH | 50.0 E-6 | — | 0.04 |
| Ascorbic | 2.2 | DPPH | 50.0 E-6 | 1.7 | 38 +/− 4 |
| Acid-2- | | | | | |
| Phosphate | | | | | |

1. Use of compatible solutes selected from the group comprised of myo-inositol phosphate (DIP), cyclic 2,3 diphosphoglycerate (cDPG), 1,1-di-glycerol phosphate (DGP), β-mannosylglycerate (firoin), β-mannosylglyceramide (firoin-A) or/and di-mannosyl-di-inositol phosphate (DMIP) and/or derivates of these compounds or combinations thereof for protecting organisms, organs, tissues, cells or the organic buildings blocks thereof from chemical radicals and oxidatively active compounds.

2. Use according to claim 1, wherein the organic building blocks are proteins, lipids, fats or nucleic acids.

3. Use according to claim 1 and/or 2, wherein the organisms are human beings, animals, and microorganisms.

4. Use according to claims 1 to 3, wherein the organs and tissues are skin, kidneys, heart, limbs.

5. Use according to at least one of claims 1 to 4, wherein the compatible solutes are used in concentrations of between 0.001 M and 2 M.

6. Method for the protection of organisms, organs, tissues, cells or the organic building blocks thereof from free radicals and oxidatively active compounds, wherein compatible solutes are added to a sample containing the organisms, organs, tissues, cells or the organic building blocks thereof.

7. Use of compatible solutes for the production of a medicine for the treatment of diseases that are caused by free radical and oxidant activity.

8. Use according to claim 7, wherein the diseases are selected from the group comprised of heart diseases such as cardiac infarction, stroke, rejection after an organ transplant, AIDS, allergies, angina, arthritis, asthma, arteriosclerosis, internal bleeding, bleeding gums, hematomas, cancer, cataracts, circulation problems, cirrhosis, diabetes type II, edemas, fatigue, hay fever, heart attack, hemorrhoids, hypertension, inflamed tissues, liver and kidney damage, impotence, memory loss, menstrual disorders, migraine, multiple sclerosis, night blindness, Parkinson's disease, phlebitis, prostate problems, psoriasis, respiratory problems, retinopathy, senility, rheumatism, skin cancer, stroke, swollen limbs, varicose veins, as well as neurodegenerative diseases, such as Huntington's disease (or HD), Parkinson's disease (or PD), Lou Gelrig's disease (or ALS), and Alzhcimer's disease (or AD).

9. Use of compatible solutes for the production of a cosmetic and/or dermatological preparation for the prevention and treatment of skin damage or skin disorders caused by free radical and oxidant activity.

10. Use according to claim 9, wherein skin damage and skin disorders are selected from the group comprised of dehydration of the skin, skin problems, dermatoses, and age spots.

11. Use of compatible solutes for the production of a food product for the prevention and treatment of damage or alteration to organisms caused by free radical and oxidant activity.
